(19) United States
(12) Patent Application Publication
(10) Pub. No.: US 2015/0157199 A1
(43) Pub. Date: Jun. 11, 2015

(54) METHOD AND APPARATUS FOR SCATTEROMETRIC MEASUREMENT OF HUMAN TISSUE

(71) Applicant: Noam Sapiens, Cupertino, CA (US)

(72) Inventor: Noam Sapiens, Cupertino, CA (US)

(21) Appl. No.: 14/099,701

(22) Filed: Dec. 6, 2013

(51) Int. Cl. A61B 3/00; A61B 3/103; A61B 3/10

(52) U.S. Cl. CPC A61B 3/0025; A61B 3/0008; A61B 3/1025; A61B 3/103; G02B 27/0927

(57) ABSTRACT

A scatterometric measurement system for measuring an object under test is disclosed. The scatterometric measurement system generates a beam of light from a light source sending the generated beam to illumination optics for transforming the beam and sending this transformed beam to a beam splitter. The beam splitter redirects the transformed beam to a first detector while deflecting the transformed light beam to the object under test which produces scattered light. Collection optics then receives this scattered light from the object under test and processes and sends the scattered light to a second detector through the beam splitter. The second detector generates a signal based on this processed scattered light and sends this result to a computation unit that calculates using the second detectors signal a desired output according to an algorithm for a given measurement for the object under test.

### CROSS-REFERENCE TO RELATED APPLICATIONS

The present application claims priority to U.S. Provisional Patent Application No. 61/733,969, filed on Dec. 6, 2012, entitled “Method and Apparatus for Scatterometric Measurement of Human Tissue” which is incorporated by reference in its entirety all having by same inventor.

### BACKGROUND

A scatterometer is a device that enables visualization of the angular, spectral, phase and/or polarization content of an object rather than its spatial, geometrical representation of that object (as usually done in imaging methods). In terms of Fourier optics, a scatterometer is based on data retrieval from a Fourier transform conjugate plane to the object rather than a conjugate plane to the object itself. Therefore, a scatterometer, according to its name measures scattered light from an object under test. This scattered light actually includes information not only on scattering in the conventional sense from the object, but rather also on diffraction (of different orders for example), absorption, reflection, transmission, and other optical qualities and their dependence on various radiation properties (e.g. direction (angle), wavelength, phase, polarization).

Typically, the measured property in a scatterometer is intensity (as is the case when using a camera). This measured result may then be processed by various algorithms according to the scatterometer setup (e.g. the use of polarizers and wave plates to detect polarization). Additionally, different optical properties may be deduced from the measurement as well as calculating other parameters for the object under test.

Most medical pathologies affect to optical properties of the affected tissue. Some of the changes manifest by increased absorption, reflection and scattering. Many changes are apparent in specific wavelengths. Current triage methods are mainly based on imaging (e.g. X-ray, OCT, tomography, microscopy etc.), namely creation of a visual representation of the affected tissue. A scatterometer measures the optical properties described above as a whole, without creating an image. Nevertheless, a scatterometer generates a distribution of the said properties that enables deduction of a myriad of parameters otherwise undetectable. Light scatter from different body parts, especially the human eye and retina can be measured by commercially available products. These use the patient subjective response to measure only the apparent stray light in the eye and is mainly only used as a cataract quantifier.

What is needed is a method and apparatus that measures a “fingerprint” signature signal from the measured object (e.g. the human eye or retina) wherein the signal from every person is expected to be unique and wherein the measurement may be done from afar.

### SUMMARY

A scatterometric measurement system for measuring an object under test is disclosed. The scatterometric measurement system generates a beam of light from a light source sending the generated beam to illumination optics for transforming the beam and sending this transformed beam to a beam splitter. The beam splitter redirects the transformed beam to a first detector while deflecting the transformed light beam to the object under test which produces scattered light. Collection optics then receives this scattered light from the object under test and processes and sends the scattered light to a second detector through the beam splitter. The second detector generates a signal based on this processed scattered light and sends this result to a computation unit that calculates using the second detectors signal a desired output according to an algorithm for a given measurement for the object under test.

### BRIEF DESCRIPTION OF THE DRAWINGS

For a clearer understanding of the invention and to see how the same may be carried out in practice, a preferred embodiment will now be described, by way of non-limiting example only, with reference to the accompanying drawing, in which:

FIG. 1 is a block-diagram of the scatterometric measurement system in accordance with the present invention;

FIG. 2 is a block diagram using the scattometeric measurement system shown in FIG. 1 for performing an eye examination;

FIG. 3 is a block diagram using the scattometeric measurement system shown in FIG. 2 for performing an eye examination incorporating a refractometer;

FIG. 4 is a block diagram using the scattometeric measurement system shown in FIG. 2 for performing an eye examination incorporating an SLO detector; and

FIG. 5 is a block diagram using the scattometeric measurement system shown in FIG. 2 for performing an eye examination incorporating a wavefront sensor and feedback system.

### DETAILED DESCRIPTION

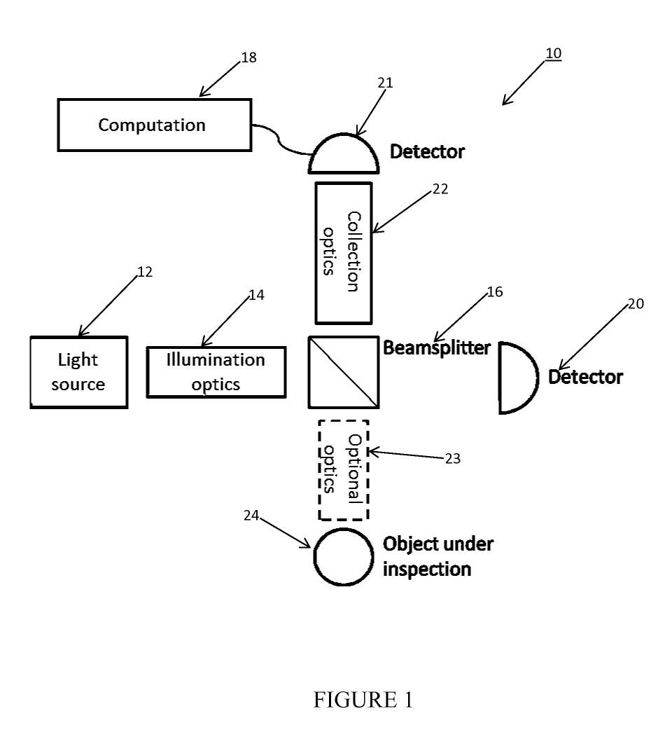

Referring now to FIG. 1 there is shown a block-diagram of a scatterometric measurement system 10 having a light source 12, illumination optics 14, beamsplitter 16, detectors 20 and means for computation 18 for measuring an object under test or inspection 24. The light source 12 may be a lamp, a laser, a super-continuum laser, a battery of lasers etc., wherein any of these different light sources may produce depending on the measurement test being performed delivers either a continuous or pulsed beam of light for processing through the illumination optics 14.

Referring once again to FIG. 1, the illumination optics 14 receives the beam of light and transforms the beam by performing optical amplitude shaping for the beam and in addition may perform polarization control, spatial control, angular control, phase control, spectral control for the same beam. It should be understood that the illumination optics 14 may be placed in a field conjugate plane (referred to as “object conjugate”), a pupil conjugate plane (which is the Fourier transform of an object plane), or anywhere in between. Therefore, any combination may be possible depending on the type of measurement being performed.

By way of example and not of limitation the optical amplitude beam shaping may be performed by any known number of techniques such as utilizing apertures, apodizers, spatial light modulators or filters (e.g to control overall power—this may also be achieved by cross polarization techniques). The polarization control may also performed by any known number of techniques such as utilizing polarizers (linear, circular, elliptic, radial/tangential), waveplates, nematic liquid crystals or other any known prior art spatial polarization controllers. The angular control may be performed by magnification optical techniques using apertures, spatial light modulators or apodizers. If phase control is needed as part of the measured data required to be collected, phase modulators may be used (e.g. electrooptic, acoustoopticoptical path modifiers (e.g. glass plates of various thicknesses, wedges on a translation stage, window on a rotation stage) or spatial phase modulators (e.g. liquid crystals). Lastly, if spectral control of the beam is needed than filters or spectral shapers may be used (e.g. a combination of a grating with a spatial light modulator that enables specific on the fly (e.g. in closed loop) tailoring of the optical spectrum). Spectral control may also be performed using shutters (when a battery of lasers is used—these can control which are used at a specific measurement).

Turning once again to FIG. 1, the beam splitter 16 receives the processed beam of light from the illumination optics 14 and directs this light to the object under inspection 24 through optional optics 23 that may include for example an objective lens (not shown). The beam splitter 16 also enables light from the illumination optics 14 to go into a first detector 20 and additionally pass through collection optics into a second detector 21. The first and second detectors 20 and 21 respectively, may be any of the following: a power meter, energy meter (when a pulsed light is delivered), a camera, or a field detection system such as a Hartman-Shack sensor. The first detector may be used for illumination beam monitoring when a modeling algorithm is needed for producing measurement results. The first detector 20 may also be used for power monitoring for safety reasons or to enable closed loop operation with the illumination optics 14 (e.g. as in adaptive optics system) that shapes the illumination according to specified criteria.

The object under inspection 24 may be any type of tissue or sample that requires testing. The collection optics 23 includes all required optics to complete a measurement test according to specific measurement metrics which may be by way of example only any of the following metrics: amplitude shaping (for example apodization of different types in either field plane (object plane) or pupil plane (Fourier transform plane)), phase control, angular control, spatial control (e.g. a collection field stop), polarization control (e.g. a polarizer for cross polarization measurement), spectral control (e.g. a grating to separate the spectrum). It should be understood that all the components that were mentioned with regards to the illumination optics 14 may also all be used here as well, along with any other known prior art components. Lastly, the second detector 21 transfers the signal received from the beam splitter 16 through the collection optics 22 into a computation unit that calculates the require output according to an algorithm for a given measurement test.

### Eye Examination

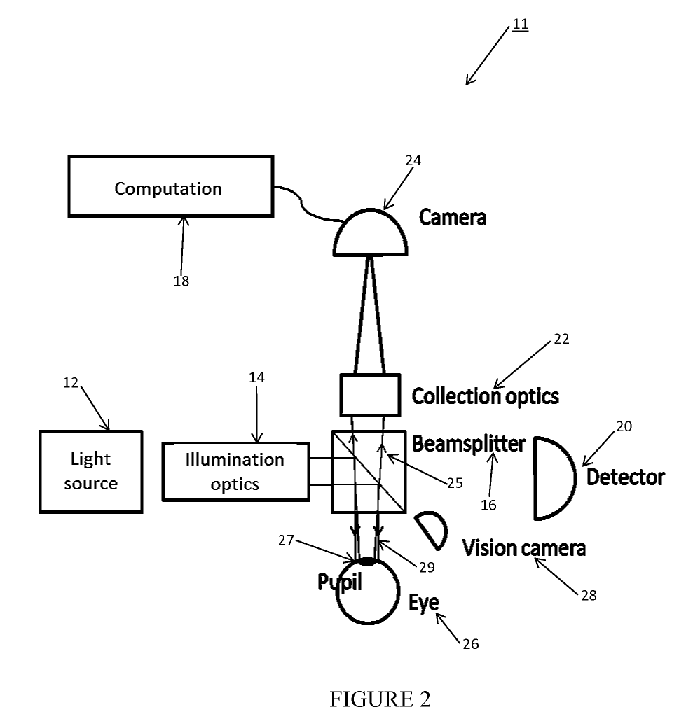

Referring now to FIG. 2 there is shown a block diagram for the scatterometeric measurement system of FIG. 1 used for performing an eye examination. In accordance with a preferred embodiment of the invention, a human eye is the most suitable organ for using the scattometeric measurement system of FIG. 1. This is due in part that an eye examination is the type of measurement test that may be non-intrusively performed using an optical system. Furthermore, it shows the most promise in a variety of test applications when it comes to this organ. Referring once again to FIG. 2, the following scatterometeric system 11 shows a simple measurement of the angular distribution of the scattering and reflection from the human eye 26 and especially the retina. Therefore, FIG. 2 illustrates one example using the invention for the triage of eye disease.

As shown in FIG. 2, the light source 12 with respect to an eye test may use any one of the following device(s): a laser, a set of lasers, a supercontinuum laser, a lamp or a lamp with different filters for transmitting a beam to the illumination optics 14. As previously described, the illumination optics shapes the beam to be either uniformly distributed, Gaussian or other known prior art shapes of intensity and phase. The polarization state may also be controlled. The beam is made such that it covers a known portion of the eye's pupil 27, particularly the entire pupil. The direct illumination beam goes into the first detector 20 that is used to monitor power delivered by the light source 12 (i.e. a laser) for further analysis and for safety reasons.

Turning once again to FIG. 2, a vision camera 28 is directed at the eye 26 to measure pupil size during an eye examination test. It could be done by various means e.g. placing a ruler next to the eye or using geometrical calculations. The vision camera 28 may also be used to determine the pupil location and orientation and include a light source that does not interfere with the test itself (e.g. an infrared LED light). The deflected beam 29 from the beam splitter 16 enters the eye 26 (which optics uses as an objective for the collimated input beam) and is reflected/scattered from it. The eye 26 is held at a specific location and orientation by using for example a head and chin rest (not shown). The returned signal 25 is then brought into the collection optics 22 that consists of a focusing element such as a lens (that may by example be either achromatic, a concave mirror or a parabolic mirror).

As stated before, filters may also be included in the collection optics 22, wherein said filters may include spectral or spatial filters, apertures or stops. For an eye examination in accordance with the invention, the second detector is a camera 24 placed at the focus plane of this element to read the signal. The camera 24 is connected to a computation unit that uses special algorithms as described before to compute the desired outcome. An example here would be a comparison to a database of known signals for different pathologies. Another example would be to use an eye model to find the main tissues that cause the signal to be as it is measured. The computation includes all data collected from the measurement including but not limited to: a signal from the vision camera 28, a signal from the first detector 20, an input illumination profile (not shown), a signal from the main camera 24 or knowledge and pre-measurement of the scatterometeric measurement system 11 properties, etc.

In some instances it may be important to differentiate the signal from different parts of the eye, for example the reflection from the cornea. This may be done by optical means in the collection optics 22 (e.g. filters or plates), by indirect measurement and computation (for example separate measurement of the cornea and subtraction of the measurement from the given signal, or by use of different optical parameters for measurement (e.g. use of different wavelengths for reducing or eliminating corneal effects). In this case the measurement may be done for a single wavelength or for a multitude of wavelengths either sequentially or simultaneously. Further information may be derived from the spectral response of the device. Another option would be to use “white” light as the light source 12 and replace the main camera 24 with a spectrometer to determine the spectral distribution of the signal. In this case the illumination optics 14 might also include apertures and other optical devices to determine the spatial and angular content of the input signal to the eye 26 and the collection optics 22 might also include such apertures and other optics to choose from the signals the desired portions (angular or spatial) to be measured.

It should be appreciated that using the scatterometeric measurement system 11 shown in FIG. 2, it is important to have control of several parameters wherein three of the most important are the pupil size of the eye (affected by ambient light, age, different illnesses/pathologies, treatments of different types e.g. pupil dilation drops), the angle at which the patient is looking or at which the illumination light enters the eye and the accommodation state of the human lens. The latter two may be controlled by placing accommodation targets (e.g. concentric circles, this target may also be made in a way that it glows in the dark when a dark measurements are required) at different distances and locations (lateral—these will convert into angles).

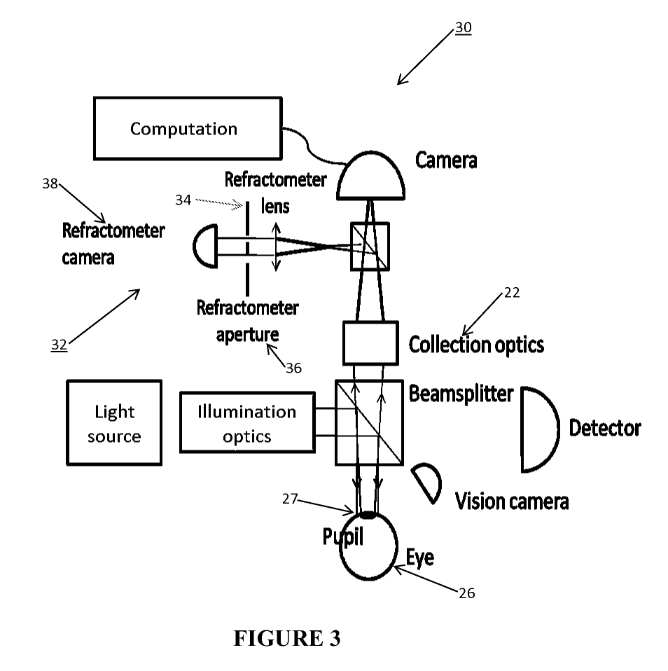

Referring now to FIG. 3 there is shown a block diagram for the scatterometeric measurement system 30 of FIG. 2 used for performing an eye examination by incorporating a refractive power measurement system 32 (referred to as a “refractometer”) into the system 30 for the accommodation measurement. A tilting mechanism (not shown) to control the angle of incidence of the illumination light upon the pupil 27 may also be incorporated. Turning once again to FIG. 3, refractometer 32 is incorporated into the scatterometeric measurement system 30 as follows: A specific light source may be used or the measurement light source 12 could be used. The beam from the collection optics 22 is split to the refractometer part of the system. It passes through a refractometer lens 34 (or other collimating optics (e.g. concave mirror, parabolic mirror) and through a refractometer aperture 36. This aperture 36 is placed in a plane conjugate to that of the eye pupil. A refractometer camera 38 then uses the distance between the two generated spots to measure the refractive power of the eye 36. For an emmetropic eye the center of the two spots is the same as the distance between the two holes in the refractometer aperture 36 since it is expected that the beam will be parallel. For hyperopia, the distance between the spots will increase and for myopia the distance will decrease. Using ray tracing techniques and the geometry of the scatterometeric measurement system 30 enables the determination of the optical power of the eye 26.

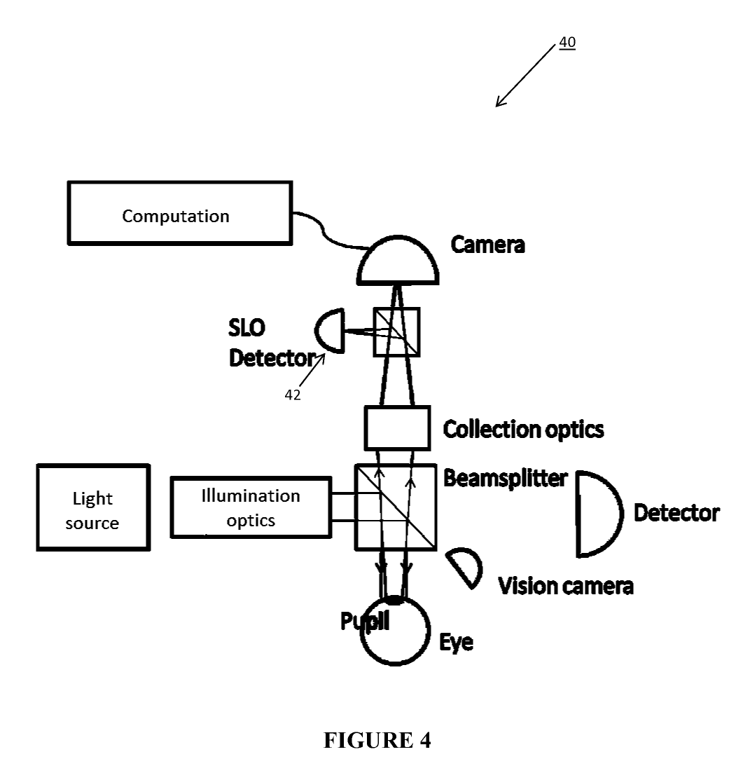

Referring now to FIG. 4 there is shown a block diagram of another preferred embodiment for the scatterometeric measurement system 40 of FIG. 2 used for performing an eye examination by incorporating a scanning laser ophthalmoscope (SLO) 42 since it could benefit from the scanning capabilities of such a system (measurement of different locations of the retina). Also, the scatterometeric measurement system 11 shown in FIG. 2 incorporates into an SLO system 40 in a relatively simple way. Here the beam scans the retina (by entering the eye at different angles) and the data received on the SLO detector 42 is used as the SLO signal.

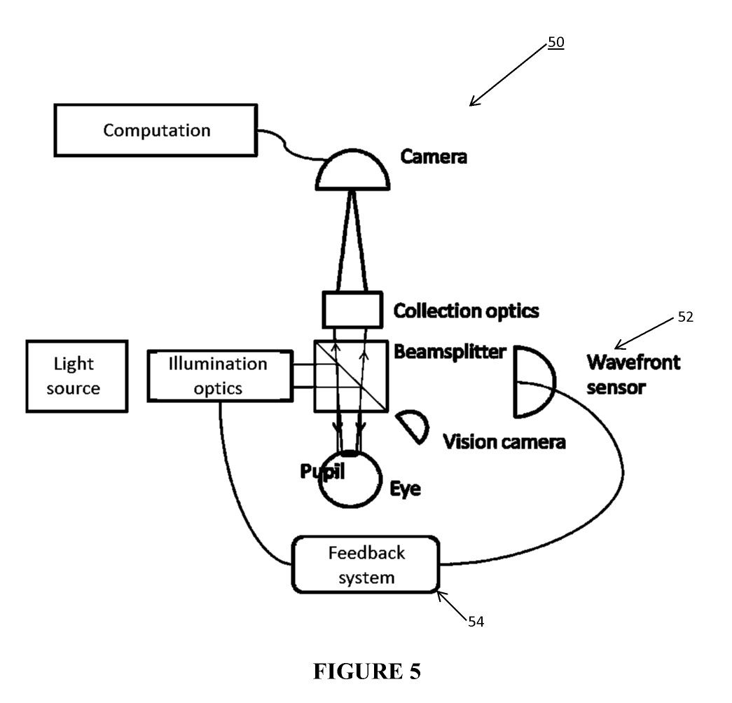

Referring now to FIG. 5 there is shown a block diagram of yet another preferred embodiment for a scatterometeric measurement system 50 used for performing an eye examination by incorporating an adaptive optics system into the scatterometeric measurement system 11 of FIG. 2. The scatterometeric measurement system 50 cancels wavefront aberration that might be due to imperfections in the optics of the system or the optics of the eye. This will enable direct measurement of the retina itself without contribution from other optical elements. There is a risk though here that the correction might cause the signal to be distorted and not completely describe the actual status of the retinal tissue. Here the first detector is replaced (or in most cases it will be used in conjunction) with a wavefront sensor 52 (e.g. a Hartmann-Shack sensor) wherein the wavefront distortion is measured—this may be done by an auxiliary light source dedicated for this purpose or by the scatterometer light source. The wavefront signal is then processed by a feedback system 54 that is connected to a SLM in the illumination optics 14 (e.g. a deformable mirror or MEMs system). The feedback is processed until a defined distortion level or structure is achieved. The designed illumination is then used as the illumination for the scatterometric measurement.

The measured signal may be compared to a population-wide standard for detection of different anomalies. Another option would be to compare the measured signal to a modeled signal according to some models of the tested tissue with specific qualities and quantities that will help detect abnormalities. Lastly, a third option would be to compare the tested signal to a library of signals (either measured or modeled) and find the most suitable anomaly resulting from the library comparison. In summary, use of scatterometry for triage benefits from all the properties of optical imaging such as the use of different wavelengths, different polarizations, and different phase and amplitude of the optical signal. The use of medical scatterometry may be applied to any tissue in the human body (or other) (permitting a suitable wavelength that can reach it). It should be noted that eyes and retinas are of particular suitability for the method of the present invention due to their transmission in the visible and near IR regions of the spectrum.

What is claimed is:

1. A scatterometric measurement system for measuring an object under test, comprising:
a light source for generating a beam of light;
illumination optics for transforming said beam of light;
a beamsplitter for redirecting said transformed beam to a first detector and deflecting said transformed beam to the object under test;
collection optics for receiving scattered light from said object under test through said beamsplitter and producing a measured optical result;
a second detector for receiving said measured optical result from said collection optics and generating a measured signal; and
a computation unit that calculates using said second detectors measured signal a desired output according to an algorithm for a given measurement for the object under test.

2. The system according to claim 1, wherein said light source is a device selected from a group consisting of: a lamp, a laser, a super-continuum laser and a battery of lasers.

3. The system according to claim 1, wherein said beam of light generated by said light source may be pulsed or continuous depending on said given measurement.

4. The system according to claim 1, wherein said illumination optics transforms the beam by performing optical amplitude shaping for said beam of light and in addition performs additional transformations selected from a group consisting of: polarization control, spatial control, angular control, phase control and spectral control for said beam of light.

5. The system according to claim 4, wherein said optical amplitude beam shaping may be performed by techniques utilizing a device selected from a group consisting of: apertures, apodizers, spatial light modulators and filters.

6. The system according to claim 4, wherein said polarization control is performed by utilizing a device selected from a group consisting of: linear polarizers, circular polarizers, elliptic polarizers, radial/tangential polarizers, waveplates, nematic and liquid crystals.

7. The system according to claim 4, wherein said angular control is performed by magnification optical techniques performed by techniques utilizing a device selected from a group consisting of: apertures, spatial light modulators and apodizers.

8. The system according to claim 4, wherein said phase control is performed by utilizing a device selected from a group consisting of: electrooptic path modifiers, acoustoopticoptical path modifiers and spatial phase modulators.

9. The system according to claim 4, wherein said spectral control is performed by utilizing a device selected from a group consisting of: filters, spectral shapers and a battery of lasers.

10. The system according to claim 1, wherein said first and second detectors is a device selected from a group consisting of: a power meter, energy meter, a camera, and a field detection system.

11. The system according to claim 1, wherein said first detector is a wave sensor used for power monitoring for safety reasons and enables closed loop operation with the illumination optics for shaping beam illumination according to specified criteria.

12. The system according to claim 1, wherein a scanning laser ophthalmoscope further processes said measured optical result between said collection optics and said second detector.

13. The system according to claim 1, wherein a refractometer further processes said measured optical result between said collection optics and said second detector.

14. The system according to claim 1, wherein said collection optics includes all required optics to complete a measurement test according to specific measurement metrics selected from a group consisting of: amplitude shaping an object plane, amplitude shaping a pupil plane, phase control, angular control, spatial control, polarization control and spectral control.

15. A method for measuring an object under test using scatterometric measurement, the method comprising the steps of:
generating a beam of light from a light source;
transforming said beam of light through illumination optics;
redirecting said transformed beam to a first detector using a beam splitter;
deflecting said transformed beam using said beam splitter to the object under test;
producing scattered light from the object under test resulting from the deflected transformed beam;
collecting said scattered light from the object under test through said beam splitter to collection optics producing an optical measured result;
sending said optical measured result to a second detector for generating a measured signal;
transmitting said measured signal to a computation unit; and
calculating a desired output from said computation unit according to an algorithm for a given measurement for the object under test using said second detectors measured signal.

16. The method according to claim 15 further comprising the step of:
calculating said desired output is by comparing said measured signal to a population-wide standard for detection of different anomalies.

17. The method according to claim 15 further comprising the step of:
calculating said desired output is by comparing said measured signal to a modeled signal derived from tested tissue models having specific qualities and quantities for detecting abnormalities.

18. The method according to claim 15 further comprising the step of:
calculating said desired output is by comparing said measured signal to a library of signals for determining the most suitable anomaly resulting from said library comparison.

19. A method for an eye examination using scatterometric measurement, the method comprising the steps of:
generating a beam of light from a light source;
transforming said beam of light through illumination optics;
redirecting said transformed beam to a first detector using a beam splitter;
deflecting said transformed beam using said beam splitter to the eye;
producing scattered light from the eye resulting from the deflected transformed beam;
collecting said scattered light from the eye through said beam splitter to collection optics producing an optical measured result;
sending said optical measured result to a second detector for generating a measured signal;
transmitting said measured signal to a computation unit; and
calculating a desired output from said computation unit according to an algorithm for a given measurement for eye using said second detectors measured signal.

20. The method according to claim 19, further comprising the step of producing an illuminated beam that covers an entire portion of the eye's pupil.
